(19) United States
(12) Patent Application Publication
(10) Pub. No.: US 2025/0067644 A1
(43) Pub. Date: Feb. 27, 2025

(54) FRACTURING EFFECT DETERMINATION METHOD AND DEVICE FOR PHASE TRANSITION FRACTURING DEEP ROCK

(71) Applicant: NORTHEASTERN UNIVERSITY, Shenyang (CN)

(72) Inventors: Zaobao LIU, Shenyang (CN); Houyu WANG, Shenyang (CN); Xin WANG, Shenyang (CN); Yulong ZHANG, Shenyang (CN); Ming WU, Shenyang (CN); Lin CHEN, Shenyang (CN); Ziang LI, Shenyang (CN); Yu ZHANG, Shenyang (CN); Yu QIN, Shenyang (CN); Chao LIN, Shenyang (CN)

(73) Assignee: NORTHEASTERN UNIVERSITY, Shenyang (CN)

(21) Appl. No.: 18/792,609

(22) Filed: Aug. 2, 2024

(30) Foreign Application Priority Data
Aug. 23, 2023 (CN) 202311065392.5

(51) Int. Cl. G01N 3/06; G01N 3/08; G01N 33/24

(52) U.S. Cl. CPC G01N 3/06; G01N 3/08; G01N 33/24; G01N 2203/0067

(57) ABSTRACT

A fracturing effect determination method for phase transition fracturing deep rock, comprising: obtaining initial data of a true triaxial rock of a fracture hole; fracturing true triaxial rock and obtaining acoustic emission information in the fracturing process; obtaining test data of the true triaxial rock after fracturing; inputting initial data and test data into a preset formula to generate test data; determining quantitative data of fracturing according to acoustic emission information and test data and determining the fracturing effect of true triaxial rock according to the quantitative data of fracturing. The method is implemented using a device comprising a carbon dioxide fracturing device, a true triaxial loading device, a fracturing starter, a storage tank, and a liquid filling device. The device is configured to achieve the quantitative optimization effect of the technical parameters required for the prevention and control measures of energy release in deep engineering rock mass

### TECHNICAL FIELD

The present invention relates to the field of rock mechanics laboratory test and gas blasting technology, in particular to a fracturing effect determination method and device for phase transition fracturing deep rock.

### BACKGROUND ART

High-energy gas fracturing represented by liquid carbon dioxide is a green and environmentally friendly fracturing rock breaking technology, the use of gas fracturing technology to carry out rock mass fracturing is also a measure to actively prevent rock bursts, which can release energy in rock mass to prevent rock burst disaster to a certain extent. Therefore, all kinds of fracturing products and devices appear on the market. Still, currently, it is not possible to quantitatively evaluate the fracturing parameters such as different high-energy gas filling amounts and shear slices with different blasting pressures, and gas fracturing under different geological stress conditions, to ensure the fracturing effect and engineering stability of deep rock engineering disaster protection.

### SUMMARY

### (1) Technical Problems to be Solved

In view of the above shortcomings and deficiencies of the existing technology, the present invention provides a fracturing effect determination method and device for phase transition fracturing deep rock, which solves the current failure to quantitatively evaluate the fracturing parameters such as different high-energy gas filling amounts and shear slices with different blasting pressures, and gas fracturing under different geological stress conditions, and ensures the fracturing effect and engineering stability technical problems of deep rock engineering disaster protection.

### (2) Technical Scheme

In order to achieve the above objective, the main technical schemes used in the present invention comprise:

In a first aspect, embodiments of the present invention provide a fracturing effect determination method for phase transition fracturing deep rock, comprising:
obtaining initial data of a true triaxial rock of a fracture hole;
fracturing true triaxial rock and obtaining acoustic emission information in the fracturing process;
obtaining test data of the true triaxial rock after fracturing according to the true triaxial rock after fracturing;
inputting initial data and test data into a preset formula to generate test data;
determining quantitative data of fracturing according to the acoustic emission information and test data, and determining the fracturing effect of true triaxial rock according to the quantitative data of fracturing.

Optionally, before obtaining the initial data of the true triaxial rock of the fracture hole, further comprising:
preparing the true triaxial rock with the fracture hole.

Optionally, preparing the true triaxial rock with the fractured hole, specifically comprising:
the fracture hole is located in a center of the true triaxial rock, and the axis of the fracture hole is parallel to a loading direction of a minimum principal stress.

Optionally, the step of fracturing the true triaxial rock and obtaining acoustic emission information in the fracturing process, comprising:
carrying out a stress loading on true triaxial rock;
fracturing the true triaxial rock after stress loading, and obtaining the acoustic emission information Uᵣ in the fracturing process.

Optionally, the initial data comprises a mean value of an initial wave velocity v_{i,0} of the true triaxial rock and a number of cracks in an initial fracture hole N₀.

Optionally, the test data comprises a mean value of wave velocity v_{i,1} of the true triaxial rock after fracturing and a number of cracks in a fracture hole N₁ after fracturing.

Optionally, the preset formula is

$$λ = \overline{( \sum \frac{{v}_{i , 1} - {v}_{i , 0}}{{v}_{i , 0}} )} , η = \frac{{N}_{1} - {N}_{0}}{{N}_{0}} ,$$

where, λ is a damage rate of wave velocity, v_{i,0} is a mean value of initial wave velocity of true triaxial rock, v_{i,1} is a mean value of wave velocity of true triaxial rock after fracturing, η is a degree of crack development in the fracturing hole, N₀ is a number of cracks in the initial fracturing hole, N₁ is a number of cracks in the fracturing hole after fracturing.

In a second aspect, embodiments of the present invention provide a device for phase transition fracturing deep rock, for implementing the fracturing effect determination method for phase transition fracturing deep rock of any of the above aspects, comprising:
a true triaxial loading device, the inside of the true triaxial loading device is used for bearing the true triaxial rock;
a carbon dioxide fracturing device, the carbon dioxide fracturing device is arranged on the surface of the true triaxial loading device near the true triaxial rock;
a fracturing starter, which is connected to the carbon dioxide fracturing device for controlling the start of the carbon dioxide fracturing device;
a storage tank, which is used for storing liquid carbon dioxide;
a liquid filling device, wherein the storage tank is connected to the true triaxial loading device via the liquid filling device, which is used to make the liquid carbon dioxide in the storage tank enter the carbon dioxide fracturing device.

Optionally, the device for phase transition fracturing deep rock, further comprising:
an acoustic emission detection sensor, which is arranged on the true triaxial rock.

Optionally, the carbon dioxide fracturing device is integrally connected to the true triaxial loading device.

### (3) Beneficial Effects

The beneficial effect of the present invention is that a fracturing effect determination method for phase transition fracturing deep rock, by obtaining the initial data of the true triaxial rock of the fracturing hole, the acoustic emission information in the fracturing process and the test data of the true triaxial rock after fracturing, the initial data and test data are input into the preset formula to generate test data, and then the quantitative data of the fracturing effect are determined according to the acoustic emission information and the test data, compared with the existing technology, it can help to carry out the quantitative optimization of the technical parameters required for the energy release prevention and control measures of deep engineering rock mass, and ensure the fracturing effect and engineering stability of deep rock engineering disaster protection.

### BRIEF DESCRIPTION OF DRAWINGS

FIG. 1 is a flow chart of a fracturing effect determination method for phase transition fracturing deep rock according to an embodiment provided in the present application;

FIG. 2 is a schematic diagram of a device for phase transition fracturing deep rock according to another embodiment provided in the present application.

### DESCRIPTION OF DRAWING MARKS

100—a true triaxial loading device, 200—a fracturing starter, 300—a storage tank, 400—a liquid filling device, 500—an acoustic emission detection sensor, 600—a carbon dioxide fracturing device, 700—a true triaxial rock.

### DETAILED DESCRIPTION

In order that the foregoing may be better understood, exemplary embodiments of the present invention will now be described in more detail, with reference to the accompanying drawings. While the drawings show exemplary embodiments of the present invention, it should be understood that the present invention may be embodied in various forms and should not be construed as limited to the embodiments set forth herein. Rather, these embodiments are provided so that this present invention will be thorough and complete, and will fully convey the scope of the present invention to those skilled in the field.

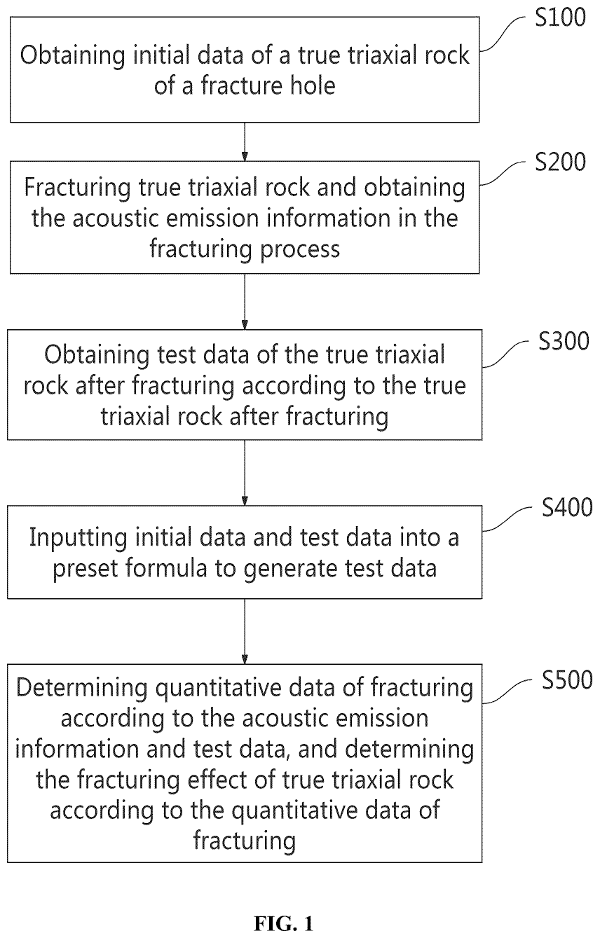

In the first aspect, as shown in FIG. 1, the present application provides a fracturing effect determination method for phase transition fracturing deep rock, comprising:
S100, initial data of a true triaxial rock of a fracture hole is obtained;
in step S100, the above initial data comprises a mean value of an initial wave velocity v_{i,0} of the true triaxial rock 700 and a number of cracks in an initial fracture hole N₀; the initial data determination and experimental preparation of true triaxial rock 700 before fracturing are carried out, the initial data determination comprises the determination of the initial wave velocity of true triaxial rock 700 and the camera observation of the borehole wall of the fracturing hole; the experimental preparation comprises the arrangement of acoustic emission monitoring and the filling and placement of the fracturing device;
wherein, the initial data determination before fracturing, the initial wave velocity determination specifically involves the three-direction six-sided cube true triaxial rock 700, and the corresponding surfaces in each direction are used array to divide equally into multiple pairs of measuring points, and the wave velocity meter is used to carry out the wave velocity determination, and the mean value of the initial wave velocity v_{i,0} is calculated; the camera observation of the borehole of the fracture hole involves the use of an endoscope to obtain the image of the hole wall, and the distribution of the cracks on the borehole wall of the rock before fracturing and the number of initial cracks N₀ are obtained;
the arrangement of acoustic emission monitoring adopts an array arrangement of acoustic emission sensors; the filling and placement of the fracturing device specifically involve filling the fracturing device with liquid carbon dioxide filling device, and then the filled liquid carbon dioxide fracturing device is placed in the fracturing channel of the true triaxial rock 700 and plugging it.

Before obtaining the initial data of the above true triaxial rock 700 of the fracture hole, further comprising: the above true triaxial rock 700 with the fracture hole is prepared; prepared the above true triaxial rock 700 with the fractured hole, specifically comprising: the above fracture hole is located in the center of the above true triaxial rock 700, and the axis of the above fracture hole is parallel to the loading direction of a minimum principal stress; the true triaxial rock 700 with the fracture hole is a standard cube sample, the parallelism error, dimension error and cross-section perpendicularity of the sample section should be in accordance with the ISRM rock experiment recommendations and procedures;
S200, the above true triaxial rock 700 is fractured and the acoustic emission information is obtained in the fracturing process;
in step S200, exemplarily, Uris the acoustic emission information.

Stress loading is carried out on the prepared true triaxial rock 700, the minimum principal stress, intermediate principal stress and maximum principal stress are applied according to the in-situ stress occurrence environment of the deep engineering, wherein, the loading direction of the minimum principal stress is parallel to the axis of the fracture hole, after the stress in three directions is applied to the stress load level, the whole process of the test remains unchanged;
the step of the above true triaxial rock 700 is fractured and acoustic emission information is obtained in the fracturing process, comprising: the stress loading on true triaxial rock 700 is carried out; the above true triaxial rock after stress loading is fractured, and the acoustic emission information Uᵣ is obtained in the fracturing process.

When the rock fracturing experiment is carried out, the acoustic emission signal in the fracturing process is monitored, according to the principle of liquid carbon dioxide phase transition, the heating device is started, and the fracturing device placed in the fracturing hole is detonated to realize the gas phase transition fracturing rock, the rock fracture signal is monitored in the fracturing process, and the acoustic emission energy Uᵣ released by the rock fracture in the fracturing process is calculated.
S300, the test data of the above true triaxial rock 700 after fracturing is obtained according to the above true triaxial rock 700 after fracturing;
in step S300, the fracturing effect of the true triaxial rock 700 after fracturing is determined, comprising the determination of the wave velocity of the rock after fracturing and the observation of the borehole image of the crack development of the fracturing hole to obtain the mean value of wave velocity of the rock sample after fracturing and the number of cracks in the fracturing hole after fracturing; the above test data comprises the mean value of wave velocity v_{i,1} of the true triaxial rock 700 after fracturing and the number of cracks in the fracture hole N₁ after fracturing.
S400, the above initial data and test data are input into a preset formula to generate test data;
in step S400, the above preset formula is

$$λ = \overline{( \sum \frac{{v}_{i , 1} - {v}_{i , 0}}{{v}_{i , 0}} )} , η = \frac{{N}_{1} - {N}_{0}}{{N}_{0}} ,$$

where, λ is a damage rate of wave velocity, v_{i,0} is the mean value of initial wave velocity of the above true triaxial rock 700, v_{i,1} is the mean value of wave velocity of the above true triaxial rock 700 after fracturing, η is a degree of crack development in the fracturing hole, N₀ is the number of cracks in the initial fracturing hole, N₁ is the number of cracks in the fracturing hole after fracturing.
S500, the quantitative data of fracturing is determined according to the above acoustic emission information and test data, and the fracturing effect of true triaxial rock is determined according to the quantitative data of fracturing.

In the second aspect, the present application provide a device for phase transition fracturing deep rock, for implementing the fracturing effect determination method for phase transition fracturing deep rock of any of the above aspects, comprising: a true triaxial loading device 100, the inside of the true triaxial loading device is used for bearing the true triaxial rock 700; a carbon dioxide fracturing device 600, the carbon dioxide fracturing device is arranged on the surface of the true triaxial loading device 100 near the true triaxial rock 700; a fracturing starter 200, which is connected to the carbon dioxide fracturing device 600 for controlling the start of the carbon dioxide fracturing device 600; a storage tank 300, which is used for storing liquid carbon dioxide; a liquid filling device 400, the storage tank 300 is connected to the true triaxial loading device 100 via the liquid filling device 400, which is used to make the liquid carbon dioxide in the storage tank 300 enter the carbon dioxide fracturing device 600.

In this technical scheme, the true triaxial rock 700 is inside the true triaxial loading device 100 during the experiment,
exemplarily, the true triaxial loading device 100 adopts rigid loading in three directions, the true triaxial loading device 100 in each direction comprises a loading plate and a loading cylinder, and a hole with a diameter of 20 mm is reserved in the center of the three-direction loading cylinder and the loading plate, and a thread is designed on the hole near the rock specimen side of the loading plate, the thread length is 10 mm, which is used to connect to the carbon dioxide fracturing device 600.

Exemplarily, the carbon dioxide fracturing device 600 can be selected but not limited to a carbon dioxide fracturing tube.

The front end of the carbon dioxide fracturing tube is designed with a thread and a length of 10 mm, which is used to connect the true triaxial loading plate; further, the wire of the carbon dioxide fracturing device 600 is connected to the fracturing starter 200 through the loading plate and the hole of the loading cylinder, and the liquid filling pipeline is connected to the liquid filling tank through the hole; the liquid filling device and the liquid storage tank 300 are connected through an external pipeline.

According to the designed true triaxial experimental device for gas fracturing, the experiment is carried out, and the rock fracturing effect is quantitatively evaluated by the degree of rock damage before and after fracturing, the quantitative evaluation method for the fracturing effect of deep rock by gas phase transition fracturing is provided, to achieve the quantitative optimization effect of the technical parameters required for the prevention and control measures of energy release in deep engineering rock mass.

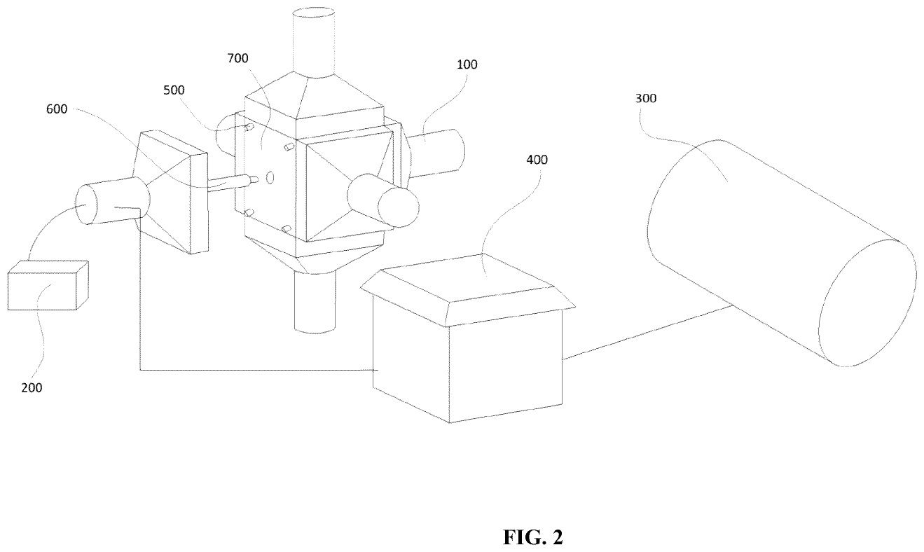

As shown in FIG. 2, further comprising: an acoustic emission detection sensor 500, which is arranged on the above true triaxial rock 700.

Exemplarily, the acoustic emission detection sensor 500 is arranged in an array on the surface of the true triaxial rock 700.

The above carbon dioxide fracturing device 600 is integrally connected to the true triaxial loading device 100.

In the description of the present invention, it is necessary to understand that the terms ‘first’ and ‘second’ are used only for descriptive purposes and cannot be understood as indicating or implying relative importance or implicitly specifying the number of technical features indicated. Therefore, the features that are limited to ‘first’ and ‘second’ can explicitly or implicitly include one or more of these features. In the description of the present invention, the meaning of ‘multiple’ is two or more, unless otherwise specified.

In the present invention, unless expressly specified and limited otherwise, terms such as ‘arranged’, ‘connected’, ‘connecting’, ‘fixed’, etc. are to be understood in a broad sense, e.g. as a fixed connection, as a detachable connection or as an integral connection; maybe a mechanical connection or an electrical connection; it can be directly connected or indirectly connected through an intermediate medium; it can be the internal connection of two components or the interaction between two components. For ordinary technicians in this field, the specific meaning of the above terms in this invention can be understood according to the specific situation.

In the present invention, unless expressly specified and limited otherwise, the first feature is ‘up’ or ‘down’ in the second feature, which can be a direct contact between the first and second features, or an indirect contact between the first and second features through an intermediate medium. Moreover, the first feature is ‘above’, ‘upward’ and ‘top’ of the second feature, which can be the first feature directly above or obliquely above the second feature, or only indicates that the first feature level is higher than the second feature. The first feature is ‘under’, ‘under’ and ‘below’ of the second feature, it can be that the first feature is directly below or obliquely below the second feature, or simply indicates that the level height of the first feature is lower than that of the second feature.

In the description of this specification, the terms ‘an embodiment’, ‘some embodiments’, ‘embodiments’, ‘examples’, ‘specific examples’ or ‘some examples’, etc. are described to mean that specific features, structures, materials or characteristics described in conjunction with the embodiment or example are contained in at least one embodiment or example of the present invention. In this specification, the indicative expression of the above terms does not have to be directed at the same embodiment or example. Moreover, the specific features, structures, materials, or characteristics described can be combined in an appropriate manner in any one or more embodiments or examples. In addition, without contradicting each other, technicians in this field can combine and combine the different embodiments or examples described in this instruction with the characteristics of different embodiments or examples.

Although the embodiments of the present invention have been shown and described above, it is understandable that the above embodiments are exemplary and cannot be understood as restrictions on the present invention. Ordinary technicians in this field can modify, modify, replace and alter the above embodiments within the scope of the present invention.

What is claimed is:

1. A fracturing effect determination method for phase transition fracturing deep rock, comprising:
obtaining initial data of a true triaxial rock of a fracture hole;
fracturing true triaxial rock and obtaining acoustic emission information in the fracturing process;
obtaining test data of the true triaxial rock after fracturing according to the true triaxial rock after fracturing;
inputting initial data and test data into a preset formula to generate test data;
determining quantitative data of fracturing according to the acoustic emission information and test data, and determining the fracturing effect of true triaxial rock according to the quantitative data of fracturing.

2. The fracturing effect determination method for phase transition fracturing deep rock according to claim 1, wherein, before obtaining the initial data of the true triaxial rock of the fracture hole, further comprising:
preparing the true triaxial rock with the fracture hole.

3. The fracturing effect determination method for phase transition fracturing deep rock according to claim 2, wherein, preparing the true triaxial rock with the fractured hole, specifically comprising:
the fracture hole is located in a center of the true triaxial rock, and the axis of the fracture hole is parallel to a loading direction of a minimum principal stress.

4. The fracturing effect determination method for phase transition fracturing deep rock according to claim 1, wherein, the step of fracturing the true triaxial rock and obtaining acoustic emission information in the fracturing process, comprising:
carrying out a stress loading on true triaxial rock;
fracturing the true triaxial rock after stress loading and obtaining the acoustic emission information Uᵣ in the fracturing process.

5. The fracturing effect determination method for phase transition fracturing deep rock according to claim 1, wherein:
the initial data comprises a mean value of an initial wave velocity v_{i,0} of the true triaxial rock and a number of cracks in an initial fracture hole N₀.

6. The fracturing effect determination method for phase transition fracturing deep rock according to claim 5, wherein:
the test data comprises a mean value of wave velocity v_{i,1} of the true triaxial rock after fracturing and a number of cracks in a fracture hole N₁ after fracturing.

7. The fracturing effect determination method for phase transition fracturing deep rock according to claim 1, wherein:
the preset formula is
$$λ = \overline{( \sum \frac{{v}_{i , 1} - {v}_{i , 0}}{{v}_{i , 0}} )} , η = \frac{{N}_{1} - {N}_{0}}{{N}_{0}} ,$$
where, λ is a damage rate of wave velocity, v_{i,0} is the mean value of initial wave velocity of true triaxial rock, v_{i,1} is a mean value of wave velocity of true triaxial rock after fracturing, η is a degree of crack development in the fracturing hole, N₀ is a number of cracks in the initial fracturing hole, N₁ is a number of cracks in the fracturing hole after fracturing.

8. A device for phase transition fracturing deep rock, for implementing the fracturing effect determination method for phase transition fracturing deep rock according to claim 1, comprising:
a true triaxial loading device, the inside of the true triaxial loading device is used for bearing the true triaxial rock;
a carbon dioxide fracturing device, the carbon dioxide fracturing device is arranged on the surface of the true triaxial loading device near the true triaxial rock;
a fracturing starter, which is connected to the carbon dioxide fracturing device for controlling the start of the carbon dioxide fracturing device;
a storage tank, which is used for storing liquid carbon dioxide; and
a liquid filling device, wherein the storage tank is connected to the true triaxial loading device via the liquid filling device, which is used to make the liquid carbon dioxide in the storage tank enter the carbon dioxide fracturing device.

9. The device for phase transition fracturing deep rock according to claim 8, further comprising:
an acoustic emission detection sensor, which is arranged on the true triaxial rock.

10. The device for phase transition fracturing deep rock according to claim 8, wherein:
the carbon dioxide fracturing device is integrally connected to the true triaxial loading device.
